(19) United States
(12) Patent Application Publication
(10) Pub. No.: US 2003/0152961 A1
(43) Pub. Date: Aug. 14, 2003

(54) Polyfunctional support material for complex nucleic acid analysis

(76) Inventors: Peter Bendzko, Berlin (DE); Uwe Schedler, Berlin (DE); Heike Matuschewski, Berlin (DE); Timo Hillebrand, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/239,396

(22) PCT Filed: Mar. 22, 2001
(86) PCT No.: PCT/DE01/01102

(30) Foreign Application Priority Data
Mar. 22, 2000 (DE) 100 13 990.6

(51) Int. Cl. C12Q 1/68; C08G 63/91; C12M 1/34

(52) U.S. Cl. 435/6; 525/54.2; 435/287.2

(57) ABSTRACT

The invention relates to a material for the complex manipulation of nucleic acids as a platform technology for developing integrative, fully automatic systems for nucleic acid analysis. The inventive support material for complex nucleic acid analysis is characterised in that at least one covalently bonded layer is located on the surface, said layer bearing at least two different functional groups which are statistically distributed on the surface. At least one of the functional groups is negatively charged and at least one other functional group is positively charged or chemically reactive or has both of these properties.

The invention relates to a material for complex manipulation of nucleic acids as a platform technology for developing integrative, fully automatic systems for nucleic acid analysis.

Potential applications in a number of molecular biology laboratories pertain to the areas of medical diagnosis, forensic diagnosis and food diagnosis, as well as all other related research fields.

The analysis of nucleic acids (RNA and DNA) becomes increasingly more important. This relates to all areas of molecular research with a biotechnological focus. A priority of the molecular nucleic acid analysis in medical research is the detection of sequence changes in medically advantageous genes, which are considered in a causal connection with pathological processes.

Based on the large amounts of target sequences that are to be examined, here the “high-throughput applications” are being increasingly accepted. Among the potential high-throughput applications, here the process solutions that are based on DNA chips (DNA arrays) are being increasingly incorporated into experimental nucleic acid analysis.

The principle of these technologies consists in that a varying number of oligonucleotides (probes) are coupled to solid phases in an addressed manner or are synthesized de novo in solid phases. The DNA chips that are produced are then incubated according to the respective problems with the sample nucleic acid that is to be analyzed. As sample nucleic acid that was to be analyzed, previously labeled total RNA was used to study expression profiles as well as labeled DNA fragments produced via PCR for sequence determination (and sequence deviation). (U.S. Pat. No. 5,837,832, U.S. Pat. No. 6,027,880)

This means that in each case corresponding to an existing requirement, DNA chips are outfitted with specific oligonucleotide samples and are hybridized below with the sample nucleic acid. The hybridization outcome is read out by means of fluorescence detection, and it is evaluated and thus yields detailed information on the existing nucleic acid sequence.

By means of this technology, it is possible to analyze a wide variety of sequence information. The extremely high costs as well as the problem of an exact quality control of the oligonucleotide samples that are addressed to a chip are disadvantageous relative to their sequence identity.

Another significant drawback of the process consists in the fact that for the analysis of nucleic acid targets with respect to a sequence detection, the targets that are to be examined must be made available. This necessity includes isolating i.R. nucleic acid from a relevant sample and subsequently amplifying the target sequences to be examined by means of PCR. Only with amplified target sequences can an addressed chip that is defined with oligonucleotides then be hybridized.

From GB 2,274,409 A, membranes are known that have structured areas that are negatively and positively charged. That is, positively and negatively charged areas are present that are delimited from one another in space. Such membranes are used as separating media in many standard filtration processes. These membranes are unsuitable, however, for a complex sequence in nucleic acid manipulation and analysis, since purification, bonding as well as detection of nucleic acids cannot occur on one site because of the spatial separation of positively and negatively charged areas.

The object of this invention was therefore to provide new support materials for a novel platform technology for complex nucleic acid manipulation and nucleic acid analysis.

This is achieved according to the invention in that previously pursued chip-based technologies are reversed from their operating principle. The invention is carried out according to the claims; the subclaims are preferential variants. The invention relates to a bifunctionalized or multifunctionalized surface on which all processes previously necessary for nucleic acid analysis (e.g. for sequence determination) are carried out.

The support material according to the invention is characterized in that at least one covalently bonded layer is found on the surface, and said layer carries at least two different functional groups that are statistically dispersed on the surface, whereby

At least one of the functional groups is negatively charged,

At least one other functional group is positively charged or chemically reactive or has two properties.

Under a statistical dispersion of the functional groups, it is understood in terms of the invention that these groups are not present isolated in space from one another but rather are found dispersed uniformly on the support surface. This has the significant advantage that all process steps can occur at one site in space of the support material.

Characteristic of the bifunctional or multifunctional solid phase is a chemical modification of the solid phase (of the substrate or support material) with at least two differently charged, in most cases ionic, functional groups or a negatively charged or positively charged functional group together with a chemically reactive group, whereby the latter makes it possible to bind the target molecule covalently after purification is completed. The process of chemical modification that is used makes it possible to adjust variably the ratio of the functional groups (cationic, anionic, capable of building up a covalent bond) that are on the corresponding surface and thus to give an exact definition of said ratio. The most important feature of the invention is the combination of a negatively charged functional group with another positively charged and/or chemically reactive functional group that makes it possible to bind the target molecule covalently and to carry out all extraction, modification and detection steps to be performed on the target molecule in the same support material. The modification of the support materials according to the invention is achieved by the treatment that is described below.

The functionalities in the support material according to the invention are generated simultaneously or in succession in any sequence by the process that is described below.

1. Production of negatively charged functional groups and then further modification with cationic functional groups (without thermally or photochemically labile functional groups)

2. Simultaneous production of negatively charged functional groups together with the cationic functionalities (without thermally or photochemically labile groups)

3. Production of negatively charged functional groups and then further modification with cationic functional groups and functionalization that follows sequentially in the third step with thermally or photochemically labile functional groups

4. Simultaneous production of negatively charged functional groups together with the cationic functionalities and then modification with thermally or photochemically labile groups

5. Simultaneous functionalization with all (anionic, cationic, and thermally/photochemically activatable) functional groups

6. Production of negatively charged functional groups and then further modification of thermally or photochemically labile functional groups

The process for the production of the support according to the invention is explained in more detail below.

To produce the strongly negative functional groups, any support material is immersed in a first process stage for the period of one minute to two hours, preferably for one hour, at room temperature in a solution of 0.1 g to 500 g of sodium hydroxide, potassium hydroxide or another strongly basic substance per 1000 g of an alcohol, preferably i-propanol. Then, the support material that is removed from the alcoholic solution is washed preferably with water until the washing solution is neutral, and it is dried, preferably for 30 minutes at 100° C.

In the subsequent second process stage, the pretreated support is coated with a photo initiator. As a solvent for the initiator, ketones, alcohols, esters and ethers are suitable. In this case, the concentration of the initiator is preferably 0.01 gl⁻¹ to 0.5 gl⁻¹.

The support material that is pretreated and charged with initiator is immersed in a monomer solution, taken from the monomer solution and exposed to the light in the moist state. As monomers, carboxylic acid, sulfonic acid and phosphoric acid derivatives that are capable of polymerization are suitable, especially the derivatives of acrylic acid, methacrylic acid, styrenesulfonic acid and styrenephosphoric acid or else mixtures thereof. As solvents for the monomers, alcohols, ketones, esters, ethers and especially water as well as mixtures thereof are used. The concentration of the monomer is preferably 1 gl⁻¹ to 200 gl⁻¹, and solutions with a monomer content of 50 gl⁻¹ are especially suitable .

As light sources, preferably mercury vapor lamps, high-pressure and maximum-pressure mercury vapor lamps, halogen lamps, tungsten lamps and lasers with emissions are used in the area of initiator absorption. The exposure time depends on the intensity of the radiation source and is a fraction of a second up to hours depending on the output thereof.

After the exposure, the support material is washed. As a washing liquid, especially the solvents that were used for the production of the monomer solution are suitable. The prepared supports are finally dried.

To produce weakly negative functional groups, the first step can be greatly shortened or omitted.

In a following third step, the previously modified support is coated in turn with a photo initiator. As a solvent for the initiator, ketones, alcohols, esters and ethers are suitable. The concentration of the initiator in this case is preferably 0.01 gl⁻¹ to 0.5 gl⁻¹ The support material that was previously modified and charged with initiator is immersed in a monomer solution, taken from the monomer solution and exposed to light in the moist state. As monomers, ammonium, sulfonium and phosphonium derivatives that are capable of polymerization are suitable, especially the derivatives of acrylic acid, methacrylic acid, styrene and vinyl compounds or else mixtures thereof. As solvents for the monomers, alcohols, ketones, esters, ethers and especially water as well as mixtures thereof are used. The concentration of the monomer is preferably 1 gl⁻¹ to 200 gl⁻¹; solutions with a monomer content of 50 gl⁻¹ are especially suitable.

As light sources, preferably mercury vapor lamps, high-pressure and maximum-pressure mercury vapor lamps, halogen lamps, tungsten lamps and lasers with emissions are used in the area of the initiator absorption. The exposure time depends on the intensity of the radiation source and is a fraction of a second up to hours depending on the output thereof.

After the exposure, the support material is washed. As a washing liquid, especially the solvents that were used for the production of the monomer solution are suitable. The prepared supports are finally dried.

In another variant embodiment, mixtures of cationic and anionic monomers are already used in photochemical modification and are polymerized at the same time. To this end, the support that is pretreated in the first step or an untreated support is coated with a photo initiator. As a solvent for the initiator, ketones, alcohols, esters and ethers are suitable. The concentration of the initiator in this case is preferably 0.01 gl⁻¹ to 0.5 gl⁻¹.

The pretreated support material or else the support material that is not pretreated and is charged with initiator is immersed in a monomeric mixture solution, taken from the solution and exposed to light in the moist state. As monomers, carboxylic acid, sulfonic acid and phosphoric acid derivatives that are capable of polymerization are suitable, especially the derivatives of acrylic acid, methacrylic acid, styrenesulfonic acid and styrenephosphoric acid or else mixtures thereof in a mixture with ammonium, sulfonium and phosphonium derivatives that are capable of polymerization.

As monomers, bifunctional or polyfunctional monomers are suitable.

As solvents for monomers, alcohols, ketones, esters, ethers and especially water as well as mixtures thereof are used. The monomer concentration is preferably 1 gl⁻¹ to 200 gl⁻¹; solutions with a monomer content of 50 gl⁻¹ are especially suitable.

As light sources, preferably mercury vapor lamps, high-pressure and maximum-pressure mercury vapor lamps, halogen lamps, tungsten lamps and lasers with emissions are used in the area of the initiator absorption. The exposure time depends on the intensity of the radiation source and is a fraction of a second up to hours according to the output thereof.

After the exposure, the support material is washed. As a washing liquid, especially the solvents that were used for the production of the monomer solution are suitable. The prepared supports are finally dried.

The application of the photo-labile or the thermally labile functional groups is carried out either as a last step after the completion of the preceding functionalizations with negative and positive functional groups or immediately after the modification with the negative functionality or together with negative and/or positive functional groups. To this end, the substrate (the support) is coated with a photo initiator. As solvents for the initiator, ketones, alcohols, esters and ethers are suitable. The concentration of the initiator in this case is preferably 0.01 gl⁻¹ to 0.5 gl⁻¹. The pretreated support material or else the support material that is not pretreated and that is charged with initiator is immersed in a monomer-mixture solution, taken from the solution and exposed to light in the moist state.

As monomers, radical generators (azides, onium salts, peroxides, nitro compounds, ketones) that are capable of polymerization or else mixtures thereof are suitable, especially aromatic bisazides or benzophenone derivatives that produce photochemically with suitable wavelength or thermal radicals and thus produce a covalent bonding to the already ionically bonded nucleic acid.

As solvents for the monomers, alcohols, ketones, esters, ethers and especially water as well as mixtures thereof are used. The monomer concentration is preferably 1 gl⁻¹ to 200 gl⁻¹; solutions with a monomer content of 50 gl⁻¹ are especially suitable.

As light sources, preferably mercury vapor lamps, high-pressure and maximum-pressure mercury vapor lamps, halogen lamps, tungsten lamps and lasers with emissions are used in the area of initiator absorption. The exposure time depends on the intensity of the radiation source and is a fraction of a second up to hours depending on the output thereof.

After the exposure, the support material is washed. As a washing liquid, especially the solvents that were used for the production of the monomer solution are suitable. The prepared supports are finally dried.

By this layer that is found on the surface, which is produced in particular by modification of the surface toward a radical polymerization that is initiated on the surface, whereby the functionalities are produced simultaneously or in any sequence and are bonded covalently to the surface, the corresponding functionalized support materials are extremely well suited for the use for complex nucleic acid analysis.

The support materials according to the invention make possible the complex processes of sample preparation, optionally specific amplification, optionally specific manipulation and the hybridization reactions that are necessary for sequence determination as well as to be able to allow a final detection to run that is fixed in space to only one reaction surface. This is a significant advantage relative to the previously used technology of complex nucleic acid analysis, since in the previous chip processes, all necessary processes of sample preparation, amplification and optionally manipulation always run separately in space.

The reversal of the previous chip technologies is based on the fact that in contrast to the prior art, no DNA chips are generated, but rather the agent according to the invention as a solid phase immobilizes the sample nucleic acid that is to be examined.

This is achieved in that a clinically relevant sample with a cell lysis buffer system is incubated in the solid phase, and after the cell lysis of the starting material is completed, optionally with the addition of an additional buffer, the nucleic acid of the cell is bonded to the negative functional groups of the solid phase. Then, the solid phase is washed with the bonded nucleic acid, and potential inhibitors are effectively removed for subsequent processes. In the further course of the process, the covalent immobilization of the sample nucleic acid is now carried out by detachment of the nucleic acid from the negative functional groups via the incubation of the solid phase with a low-salt buffer system (e.g., 10 mmol of tris HCl). This has the result that the nucleic acids that are separated from the negative charge in turn wind up interacting with the positive functional groups of the solid phase because of this negativity. The ultimately achieved covalent bonding is carried out under chemical and photochemical generation of radicals on the surface, which in turn link covalent bonds by H-abstraction.

The now covalently immobilized nucleic acid can be manipulated in any form below. In a variant embodiment, DNA single strands are produced, e.g., by alkaline denaturation, and a target detection is performed by the hybridization process that is known to one skilled in the art with specific oligonucleotide probes, whereby the hybridization outcome is determined according to the process that is known in the prior art.

By the use of multiple probes with various detection markers, a wide variety of sequence information can then be determined, e.g., very simply. In addition, the possibility also exists of removing probes again via highly stringent washing steps to begin again the process for detecting other target sequences in the nucleic acid sample below.

It is evident from this example that basically any manipulation of nucleic acids that are advantageous for nucleic acid analysis can be carried out by means of the process according to the invention. By the integration of all processes from sample preparation (nucleic acid extraction) to the detection reaction, a solid phase results such that the complex automation of nucleic acid analysis can be easily accomplished and thus is especially advantageous.

The production of the support material according to the invention is explained in more detail below in an embodiment.

Embodiment

Membrane, Negatively and Positively Charged (Ionic)

Variant a) Sequential Modification (Negatively and Positively Charged Groups in Succession)

A polypropylene membrane in the DIN A4 format (pore size 0.2 μm) is immersed for 5 minutes in a 150 mmol solution of benzophenone (photo initiator) in acetone and then dried. Then, the membrane is covered in a glass tray with the reaction solution that consists of 20 g/l of acrylic acid in water. The glass tray is covered with a glass plate (low-UV filter, λ>310 nm). After 10 minutes, the UV dryer (Beltron Gmbh) is exposed to light at half-load for 10 minutes (10 passes through the exposure zone). Then, the membrane is extracted with methanol and water. Then, it is dried and the degree of modification is determined by gravimetry.

The membrane that is already functionalized with negative groups is immersed for 5 minutes in a 150 mmol solution of benzophenone (photo initiator) in acetone and then dried. Then, the membrane is covered in a glass tray with the reaction solution that consists of 30 g/l of 2-aminoethylmethacrylic acid amide hydrochloride in water.

The glass tray is covered with a glass plate (low-UV filter, λ>310 nm). After 10 minutes, the UV dryer (Belton GmbH) is exposed to light at half-load for 20 minutes (20 passes through the exposure zone). Then, the membrane is extracted with methanol and water. Then, it is dried and the degree of modification is determined by gravimetry.

Variant b) Simultaneous Modification (Negatively and Positively Charged Groups Simultaneously)

A polypropylene membrane in the DIN A4 format (pore size 0.2 μm) is immersed for 5 minutes in a 150 mmol solution of benzophenone (photo initiator) in acetone and then dried. Then, the membrane is covered in a glass tray with the reaction solution that consists of 20 g/l of acrylic acid and 30 g/l of 2-aminoethylmethacrylic acid amide hydrochloride in water. The glass tray is covered with a glass plate (low-UV filter, λ>310 nm). After 10 minutes, the UV dryer (Beltron GmbH) is exposed to light at half-load for 20 minutes (20 passes through the exposure zone). Then, the membrane is extracted with methanol and water. Then, it is dried, and the degree of modification is determined by gravimetry.

FTIR-ATR Spectroscopy:

1720 cm⁻¹ (C═O valence vibration) carboxyl group

1650 cm⁻¹ (C═O valence-vibration) acid amide I

1540 cm⁻¹ (N—H deformation) acid amide II

3300 cm⁻¹ (N—H valence vibration) amino group (protonated)

1. Support material for complex nucleic acid analysis, characterized in that at least one covalently bonded layer is found on the surface that carries at least two different functional groups and that is statistically dispersed on the surface, whereby
at least one of the functional groups is negatively charged,
at least one other functional group is positively charged or chemically reactive or has both properties.

2. Support material according to claim 1, wherein at least one layer that is found on the surface in addition to at least one negatively charged functional group contains at least one positively charged functional group.

3. Support material according to claim 1, wherein at least one layer that is found on the surface in addition to at least one negatively charged functional group contains at least one chemically reactive functional group.

4. Support material according to claim 1, wherein at least one layer that is found on the surface in addition to at least one negatively charged functional group contains at least one functional group that is both positively charged and chemically reactive.

5. Support material according to claim 1, wherein at least one layer that is found on the surface in addition to at least one negatively charged functional group contains at least one positively charged functional group and at least one chemically reactive functional group.

6. Support material according to claims 1 to 5, wherein at least one of the negatively and/or positively charged functional groups is an ionic functional group.

7. Support material according to claims 1 to 6, wherein the negatively charged groups that are found on the surface are formed from compounds, capable of polymerization, with carboxyl, sulfonyl, sulfate and phosphate groups, especially derivatives of acrylic acid, methacrylic acid, styrenesulfonic acid and styrenephosphoric acid, acrylamidopropanesulfonic acid and/or mixtures thereof.

8. Support material according to claims 1, 2 and 4 to 7, wherein the positively charged functional groups consist of compounds, capable of polymerization, with amino or ammonium, sulfonium or phosphonium groups, especially derivatives of acrylic acid, methacrylic acid, styrene and/or mixtures thereof.

9. Support material according to claims 1 and 3 to 8, wherein the chemically reactive functional groups can be activated chemically and/or thermally and/or photochemically.

10. Support material according to claims 1 and 3 to 9, wherein the chemically reactive functional groups consist of immobilized sensitizers such as, for example, ketones, especially benzophenones, benzoins, xanthones or aliphatic or aromatic azides.

11. Support material according to claims 1 and 10, wherein at least one layer that is found on the surface in addition contains a UV-VIS-absorption-spectroscopically active substance, especially dyes such as fluorescein, rhodamine, anthracene, pyrene or derivatives thereof.

12. Support material according to claims 1 to 11, wherein at least one layer that is found on the surface is produced by modification of the surface toward a radical polymerization that is initiated on the surface, whereby the functionalities are produced simultaneously or in any sequence and are bonded covalently to the surface.

13. Support material according to claims 1 to 12, wherein at least one layer that is found on the surface is produced by heterogenic photo-initiated graft polymerization of functional monomers.

14. Support material according to claims 1 to 13, wherein a substance of H-abstraction type is used as a photo initiator, and the support material is used as a co-initiator, and the initiation is carried out by light excitation of the photo initiator.

15. Support material according to claims 1 to 13, wherein at least one layer that is found on the surface is produced by a chemically-initiated polymerization of functional monomers on the surface.

16. Support material according to claims 1 to 15, wherein at least one layer that is found on the surface contains a substance as an initiator that produces radicals or other starter species for polymerization after physical or chemical excitation.

17. Support material according to claims 1 to 16, wherein the functionalities on the surface of the support material are obtained by thermal or photochemical activation of bifunctional azides, such as, e.g., p-azidobenzenesulfonic acid.

18. Support material according to claims 1 to 17, wherein the negative and positive functional groups are applied in succession on the support material that is pretreated with a photo initiator, especially benzophenone, especially by a successive covering of the support material, such as, e.g., a polypropylene membrane, with solutions of acrylic acid and 2-aminoethylmethacyrlic acid amide hydrochloride, with subsequent functionalization by exposure in each case.

19. Support material according to claims 1 to 17, wherein the negative and positive functional groups are applied simultaneously to the support material that is pretreated with a photo initiator, especially benzophenone, especially by a simultaneous covering of the support material, such as, e.g., a polypropylene membrane, with a reaction solution that contains acrylic acid and 2-aminoethylmethacrylic acid amide hydrochloride, with subsequent functionalization by exposure.

20. Support material according to claims 1 to 19, wherein organic polymers, such as, e.g., polypropylene, polyethylene, polysulfone, polyether sulfone, polystyrene, polyvinyl chloride, polyacrylonitrile, cellulose and derivatives thereof, polyamides, polyimides, polytetrafluororthylene, polyvinylidene difluoride, polyester, polycarbonate, polyacrylates, polyacrylamide as well as copolymers or polymer blends are used as support materials.

21. Support material according to claims 1 to 19, wherein inorganic, especially mineral materials such as glasses, silicates, ceramics or metals as well as composites thereof with organic polymers are used as support materials.

22. Support material according to claims 1 to 21, wherein membranes, films, microtiter plates, slides, fibers, hollow fibers, mats, tissues, powders, granulates or particles, in each case porous or nonporous, are used as support materials for the reaction chamber.

23. Support material according to claims 1 to 22, wherein membranes with symmetrical or asymmetrical pore structure and pore sizes between a few nanometers and 10 μm are used as support materials.

24. Support material according to claims 1 to 19, wherein combinations of the materials that are presented in claims 20 to 23 are used as support materials.

25. Support material according to claims 1 to 24, wherein the support material is additionally functionalized in advance, e.g., hydrophilized.

26. Support material according to claims 1 to 25, wherein the pretreatment of the support material in an alkaline manner is carried out with a solution that consists of a strongly basic substance, preferably alkali-hydroxides or alkali-amides, and an alcohol, preferably in I-propanol, for up to 24 hours, and the support material is then washed until the wash water is neutral, and then it is dried.

27. Use of the support material according to claims 1 to 26 for complex and automatable analysis of nucleic acids, such as for detection of mutations, for detection of methylation patterns, for detection of SNP's and/or for detection of restriction patterns.

28. Process for complex and automatable analysis of biological materials, especially nucleic acids, wherein the necessary preparation and analysis steps, especially the extraction, binding, manipulation and detection of the nucleic acids are performed simultaneously or in succession on the same support material.

29. Process according to claim 28, wherein the starting samples that are to be examined are incubated on the support material with a cell lysis buffer, optionally with the incorporation of a proteolytic enzyme, optionally are mixed with a bonding buffer after cell lysis is completed, and the nucleic acid of the starting sample is bonded to the negatively functionalized groups and then washed, and is subsequently bonded to the positively functionalized groups by adding a buffer, especially a low-salt buffer.

30. Process according to claims 28 and 29, wherein the staring samples that are to be examined are incubated in the support material with a cell lysis buffer optionally with the incorporation of a proteolytic enzyme, optionally are mixed with a bonding buffer after cell lysis is completed, and the nucleic acid of the starting sample is bonded to the negatively functionalized groups and then washed, and is subsequently bonded to the chemically reactive groups by exposure to light and/or thermal treatment.

31. Process according to claims 28 to 30, wherein the nucleic acids that are bonded to the support material are manipulated, especially denatured, chemically modified, multiplied, selectively multiplied or digested with restriction enzymes.

32. Process according to claims 28 to 31, wherein the nucleic acids that are bonded to the support material are hybridized with specific probes.

33. Process according to claims 28 to 32, wherein the nucleic acids that are bonded to the support material are hybridized after manipulation with specific probes.

34. Process according to claims 28 to 33, wherein the detection of the hybridization is carried out indirectly by enzymatic means with labeled probes that are used.

35. Process according to claims 28 to 34, wherein the detection of the hybridization is carried out directly via the labeling of the probes.

36. Test kit for analysis of biological materials, especially nucleic acids, wherein support materials according to claims 1 to 26 and/or the process according to claims 28 to 35 as well as additional reaction components are used for nucleic acid analysis.
